(19) United States
(12) Patent Application Publication
(10) Pub. No.: US 2004/0157793 A1
(43) Pub. Date: Aug. 12, 2004

(54) Modified nucleosides as antiviral agents

(76) Inventors: Lieven J. Stuyver, Snellville, GA (US); Chung K. Chu, Athens, GA (US)

Correspondence Address: MERCHANT & GOULD PC, P.O. BOX 2903, MINNEAPOLIS, MN, 55402-0903, US

(21) Appl. No.: 10/706,865

(22) Filed: Nov. 12, 2003

Related U.S. Application Data
(60) Provisional application No. 60/425,534, filed on Nov. 12, 2002.

(51) Int. Cl. A61K 31/7076; A61K 31/7072; A61K 31/675; A61K 31/52

(52) U.S. Cl. 514/47; 514/50; 514/81; 514/85; 544/243; 544/244; 544/277; 544/310; 536/26.1; 514/263.24

(57) ABSTRACT

The present invention relates to 3′ substituted-2′, 3′-didehydro-2′, 3′-dideoxy-β-L-nucleosides and their pharmaceutically acceptable salts and prodrugs thereof, for the treatment of infectious viral diseases, in general, particularly HBV and HIV viral infections and more particularly, HBV and HIV viral infections that are resistant to other antiviral drugs.

### CROSS-REFERENCE TO RELATED PATENT APPLICATION

This application claims the benefit, pursuant to 35 U.S.C. §119(e), of provisional U.S. Patent Application Serial No. 60/425,534, filed Nov. 12, 2002 entitled “SYNTHESIS OF MODIFIED FLUORINATED NUCLEOSIDE ANALOGUES,” the disclosure of which is hereby incorporated herein in its entirety by reference.

### FIELD OF THE INVENTION

The present invention relates to 3′ substituted--2′,-3′-didehydro--2′,-3′-dideoxy--β-L-nucleosides for the treatment of infectious viral diseases, in general, particularly HBV and HIV viral infections and more particularly, HBV and HIV viral infections that are resistant to other antiviral drugs.

### BACKGROUND OF THE INVENTION

The present invention relates to 3′ substituted--2′,-3′-didehydro--2′,-3′-dideoxy--β-L-nucleosides of general formula [I] or [II] and their pharmaceutically acceptable salts and prodrugs for treatment of infectious viral diseases, in general, particularly HBV and HIV viral infections and more particularly, HBV and HIV viral infections that are resistant to other antiviral drugs:

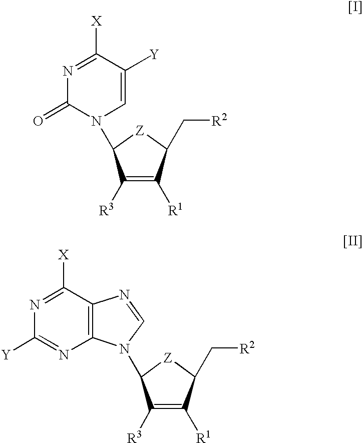

wherein:

X is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.

Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.

R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R⁴,

R² is OH, OR⁴, OC(O)R⁴, OPO₃H₂, OP₂O₆H₃, OP₃O₉H₄, OPO₃Na₂, OPO₃R⁴R⁵, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NHR⁴, NR⁴R⁵, NHOH, NHOR⁴, NHNH₂, NR⁴NH₂, NHNHR⁴, PO₃H₂, P₂O₆H₃, P₃O₉H₄, PO₃Na₂, P₂O₆Na₃, P₃O₉Na₄, PO₃R⁴R⁵, P₂O₆R⁴₂R⁵, P₃O₉R⁴₃R⁵.

R³ is H.

Z is O, S, CH₂ or C═CH₂.

wherein R⁴ and R⁵ are the same or different and are lower alkane or alkene or acyl of carbon 1-17 or aryl or aralkyl, such as unsubstituted or substituted phenyl or benzyl.

In 1981, acquired immune deficiency syndrome (AIDS) was identified as a disease that severely compromises the human immune system, and that almost without exception leads to death. In 1983, the etiological cause of AIDS was determined to be the human immunodeficiency virus (HIV). The World Health Organization estimated that by the end of 2002 that 42 million people worldwide were infected with HIV. Each day approximately 14,000 people were newly infected in 2002.

In 1985, it was reported that the synthetic nucleoside 3′-azido-3′-deoxythymidine (AZT) inhibits the replication of human immunodeficiency virus. Since then, a number of other synthetic nucleosides, including 2′,3′-dideoxyinosine (DDI), 2′,3′-dideoxycytidine (DDC), and 2′,3′-dideoxy-2′,3′-didehydrothymidine (D4T), have been proven to be effective against HIV. After cellular phosphorylation to the 5′-triphosphate by cellular kinases, these synthetic nucleosides are incorporated into a growing strand of viral DNA, causing chain termination due to the absence of the 3′-hydroxyl group. They can also inhibit the viral enzyme reverse transcriptase.

The success of various synthetic nucleosides in inhibiting the replication of HIV in vivo or in vitro has led a number of researchers to design and test nucleosides that substitute a heteroatom for the carbon atom at the 3′-position of the nucleoside (Norbeck et al. 1989, Tetrahedron Letters, 30 (46) 6246, European Patent Application Publication No. 0 337 713, and U.S. Pat. No. 5,041,449).

U.S. Pat. No. 5,047,407 and European Patent Application Publication No. 0 382 526, disclose a number of racemic 2′-substituted-5′-substituted-1,3-oxathiolane nucleosides with antiviral activity, and specifically report that the racemic mixture (about the C4′-position) of the C1′-βisomer of 2-hydroxymethyl-5-(cytosin-1-yl)-1,3-oxathiolane (i)-BCH-189) has approximately the same activity against HIV as AZT, and no cellular toxicity at the tested levels. (i)-BCH-189 has also been found to inhibit the replication of AZT-resistant HIV isolates in vitro from patients who have been treated with AZT for longer than 36 weeks. The (−)-enantiomer of the isomer of BCH-189, known as 3TC, is highly potent against HIV and exhibits little toxicity. (−)-cis-2-hydroxymethyl-5-(5-fluorocytosin-1-yl)-1,3-oxathiolane (“FTC”) also has potent HIV activity (Schinazi et al. 1992 Antimicrob. Agent and Chemotherap, 2423-2431).

Another virus that causes a serious human health problem is the hepatitis B virus (referred to below as “HBV”). HBV is second only to tobacco as a cause of human cancer. The mechanism by which HBV induces cancer is unknown, although it is postulated that it may directly trigger tumor development, or indirectly trigger tumor development through chronic inflammation, cirrhosis, and cell regeneration associated with the infection.

After a two to six month incubation period in which the host is unaware of the infection, HBV infection can lead to acute hepatitis and liver damage that causes abdominal pain, jaundice, and elevated blood levels of certain enzymes. HBV can cause fulminant hepatitis, a rapidly progressive, often fatal form of the disease in which massive sections of the liver are destroyed.

In western industrialized countries, high-risk groups for HBV infection include those in contact with HBV carriers or their blood samples. The epidemiology of HBV is very similar to that of acquired immune deficiency syndrome, which accounts for why HBV infection is common among patients with AIDS or AIDS-related complex. However, HBV is more contagious than HIV. Both FTC and 3TC exhibit activity against HBV (Furman et al. 1992 Antimicrobial Agents and Chemotherapy, 2686-2692).

A human serum-derived vaccine has been developed to immunize patients against HBV. While it has been found effective, production of the vaccine is troublesome because the supply of human serum from chronic carriers is limited, and the purification procedure is long and expensive. Further, each batch of vaccine prepared from different serum must be tested in chimpanzees to ensure safety. Vaccines have also been produced through genetic engineering. Daily treatments with α-interferon, a genetically engineered protein, has also shown promise.

In light of the fact that acquired immune deficiency syndrome, AIDS-related complex, and hepatitis B virus have reached epidemic levels worldwide, and have tragic effects on the infected patient, there remains a strong need to provide new effective pharmaceutical agents to treat these diseases and that have low toxicity to the host.

There is mounting clinical evidence that resistance to antiviral agents is a predictor for poor clinical outcome. HBV-infected patients treated with 3TC and (−)-FTC for more than 6 months face the risk of development of resistant virus. Such 3TC-resistant viruses have few, but important, amino acid changes in the conserved domains B and C of the viral polymerase. Key mutations conferring resistance to 3TC are rtM204V, rtM204I (located in the YMDD motif), and rtL180M (located in domain B; Stuyver L. J. et al. 2001, Hepatol, 33:751-757). Similar observations have been made with HIV-1, where key mutations also occur in the YMDD motif (e.g., M184V and M184I).

All currently available L-nucleoside analogues tested against HIV-1 select for the V184 mutation (Schinazi R. F. et al. 2001. Antivir. Chem. Chemother, 12:61-65). These compounds include 3TC, (−)-FTC, β-L-FDDC, β-L-D4FC and β-L-purine nucleoside analogues. In addition, a series of compounds related to β-L-D4-cytidine analogues was recently described with potent antiviral activity against both HIV-1 and HBV; in the study, the β-L-D4-analogues were found at least 586-fold less potent against a lamivudine-resistant HIV-1 viral strain (Stuyver L. J. et al. 2002, Antimicrob. Agents Chemother., 46:3854-3860).

Thus, there is a need to develop new treatments for drug resistant viral infections.

### SUMMARY OF INVENTION

The present invention relates to 3′ substituted-2′,3′-didehydro-2′,3′-dideoxy-β-L-nucleosides of the general formula [I] and [II] and their pharmaceutically acceptable salts and prodrugs for the treatment of hepatitis B (HBV) and HIV infections in a host, an, in particular, HBV and HIV infections resistant to other antiviral drugs. More particularly, the nucleosides of the present invention can be used to treat HBV and HIV infections resistant to 3TC (lamivudine).

Specifically provides are compounds of the structure:

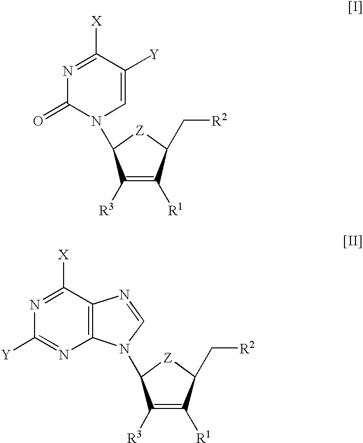

wherein:

X is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.

Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.

R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R^{4.}

R² is OH, OR⁴, OC(O)R⁴, OPO₃H₂, OP₂O₆H₃, OP₃O₉H₄, OPO₃Na₂, OPO₃R⁴R⁵, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NHR⁴, NR⁴R⁵, NHOH, NHOR⁴, NHNH₂, NR⁴NH₂, NHNHR⁴, PO₃H₂, P₂O₆H₃, P₃O₉H₄, PO₃Na₂, P₂O₆Na₃, P₃O₉Na₄, PO₃R⁴R⁵, P₂O₆R⁴₂R⁵, P₃O₉R⁴R⁵.

R³ is H.

Z is O, S, CH₂ or C═CH₂.

wherein R⁴ and R⁵ are the same or different and are lower alkane or alkene or acyl of carbon 1-17 or aryl or aralkyl, such as unsubstituted or substituted phenyl or benzyl.

In a preferred embodiment for the 3′-modified pyrimidine nucleosides, X is NH₂; Y is independently H or F; Z is 0; R¹ is F; and R² is OH. The term “independently” means that the groups can vary within the compound.

Preferred compounds include the racemic mixture, β-D and β-L isomers of the following compounds: β-L-3′-fluoro-2′,3′--didehydro-2′, 3′--dideoxycytadine(β-L-3F-D4C); and β-L-3′-fluoro-2′,3′-didehydro-2′,3′-dideoxy-5-fluorocytidine (β-L-3 F-D4FC).

In other embodiments of the present invention, the compounds and pharmaceutical compositions disclosed herein are used to treat a host with an HBV or HIV viral infection. In still other embodiments, the compounds and pharmaceutical compositions disclosed herein, are used to treat a host with an HBV or HIV viral infection that is resistant to one or more other antiviral agents.

In another embodiment, the active compound or its derivative or salt or pharmaceutically acceptable prodrug can be administered in combination or alternation with another antiviral agent, such as an anti-HIV agent or anti-HBV agent, including those described above. In general, during alternation therapy, an effective dosage of each agent is administered serially, whereas in combination therapy, an effective dosage of two or more agents are administered together. The dosages will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Nonlimiting examples of antiviral agents that can be used in combination with the compounds disclosed herein include, but are not limited to, the (−)-enantiomer of 2-hydroxymethyl-5-(5-fluorocytosin-1-yl)-1,3-oxathiolane ((−)-FTC); the (−)-enantiomer of 2-hydroxymethyl-5-(cytosin-1-yl)-1,3-oxathiolane (3TC); carbovir, acyclovir, interferon, famciclovir, penciclovir, AZT, DDI, DDC, L-(−)-FMAU, D4T, adefovir, and tenofovir.

The compounds can also be used to treat equine infectious anemia virus (EIAV), feline immunodeficiency virus, and simian immunodeficiency virus. (Wang, S. et al 1993, First National Conference on Human Retroviruses and Related Infections, 12-16; Sellon, D. C., 1993, Vet. Clin. North Am. Equine Pract. United States, 9:321-336; Philpott, M. S. et al. 1992, Vet. Immunol. Immunopathol., 35:155-166)

### BRIEF DESCRIPTION OF THE DRAWINGS

FIG. 1 depicts the concentration-dependent suppression of HBV production in HepAD38 cells. HepAD38 cells were treated for 5 days with test compounds and the reduction of viral DNA in culture supernatant was measured by Q-PCR. ♦: 3TC; ▪: L-3′F-D4C; ▴: L-3′F-D4FC.

FIG. 2 depicts the HBV viral load reduction in HepAD79 cells. Cells were seeded in presence of tetracycline, and two days post seeding the media was replaced with tetracycline-free media, but including the test compound. Subsequently, cells were incubated for an additional 13 days.

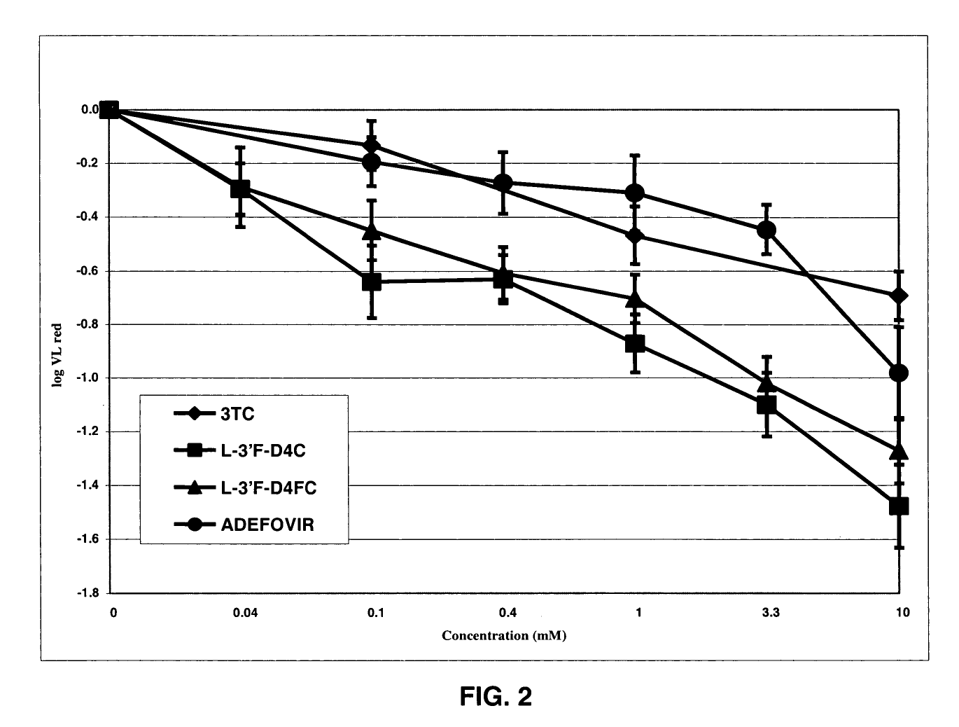

### DETAILED DESCRIPTION OF THE INVENTION

Various embodiments of the invention are now described in detail. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner in describing the compositions and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

The present invention relates to 3′ substituted--2′, 3′--didehydro--2′, 3′--dideoxy--β--L nucleosides of the general formula [I] and [II] and their pharmaceutically acceptable salts and prodrugs for the treatment of hepatitis B (HBV) and HIV infections in a host, and, in particular, HBV and HIV infections resistant to other antiviral drugs. More particularly, the nucleosides of the present invention can be used to treat HBV and HIV infections resistant to 3TC (lamivudine) and (−)-FTC.

The disclosed compounds or their pharmaceutically acceptable derivatives or salts or pharmaceutically acceptable formulations containing these compounds are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpurea and opportunistic infections. In addition, these compounds or formulations can be used prophylactically to prevent or retard the progression of clinical illness in individuals who are anti-HIV antibody or HIV-antigen positive or who have been exposed to HIV.

The compound and its pharmaceutically acceptable derivatives or pharmaceutically acceptable formulations containing the compound or its derivatives are also useful in the prevention and treatment of HBV infections and other related conditions such as anti-HBV antibody positive and HBV-antigen positive conditions, chronic liver inflammation caused by HBV, cirrhosis, acute hepatitis, fulminant hepatitis, chronic persistent hepatitis, and fatigue. These compounds or formulations can also be used prophylactically to prevent or retard the progression of clinical illness in individuals who are anti-HBV antibody or HBV-antigen positive or who have been exposed to HBV.

The compound can be converted into a pharmaceutically acceptable ester by reaction with an appropriate esterifying agent, for example, an acid halide or anhydride. The compound or its pharmaceutically acceptable derivative can be converted into a pharmaceutically acceptable salt thereof in a conventional manner, for example, by treatment with an appropriate base. The ester or salt of the compound can be converted into the parent compound, for example, by hydrolysis.

Definitions

The term “independently” is used herein to indicate that the variable, which is independently applied, varies independently from application to application. Thus, in a compound such as R^{a}XYR^{a}, wherein R^{a} is “independently carbon or nitrogen,” both R^{a} can be carbon, both R^{a} can be nitrogen, or one R^{a} can be carbon and the other R^{a} nitrogen.

As used herein, the term “enantiomerically pure” refers to a nucleoside composition that comprises at least approximately 95%, and preferably approximately 97%, 98%, 99% or 100% of a single enantiomer of that nucleoside.

As used herein, the term “substantially free of” or “substantially in the absence of refers to a nucleoside composition that includes at least 85 or 90% by weight, preferably 95% to 98% by weight, and even more preferably 99% to 100% by weight, of the designated enantiomer of that nucleoside. In a preferred embodiment, in the methods and compounds of this invention, the compounds are substantially free of enantiomers.

Similarly, the term “isolated” refers to a nucleoside composition that includes at least 85 or 90% by weight, preferably 95% to 98% by weight, and even more preferably 99% to 100% by weight, of the nucleoside, the remainder comprising other chemical species or enantiomers.

The term “alkyl,” as used herein, unless otherwise specified, refers to a saturated straight, branched, or cyclic, primary, secondary, or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Alkyl groups can be optionally substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, or any other viable functional group that does not inhibit the pharmacological activity of this compound, either unprotected, or protected, as necessary, as known to those skilled in the art, for example, as taught in Greene et al. 1991, Protective Groups in Organic Synthesis, John Wiley & Sons, 2^{nd} Edition, hereby incorporated by reference.

The term “lower alkyl,” as used herein, and unless otherwise specified, refers to a C₁ to C₄ saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The terms “alkylamino” or “arylamino” refer to an amino group that has one or two alkyl or aryl substituents, respectively.

The term “protected,” as used herein and unless otherwise defined, refers to a group that is added to an oxygen, nitrogen, or phosphorus atom to prevent its further reaction or for other purposes. A wide variety of oxygen and nitrogen protecting groups are known to those skilled in the art of organic synthesis.

The term “aryl,” as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene et al. 1991, Protective Groups in Organic Synthesis, John Wiley & Sons, 2nd Edition.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen (F, Cl, Br, I), C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group.

The term “lower acyl” refers to an acyl group in which the non-carbonyl moiety is lower alkyl.

The term “host,” as used herein, refers to a unicellular or multicellular organism in which the virus can replicate, including cell lines and animals, and preferably a human. Alternatively, the host can be carrying a part of the viral genome, whose replication or functions can be altered by the compounds of the present invention. The term host specifically refers to infected cells, cells transfected with all or part of the viral genome, and animals, in particular, primates and humans. In most animal applications of the present invention, the host is a human patient. Veterinary applications, in certain indications, however, are clearly anticipated by the present invention.

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

Synthesis of the Active Compounds

1. Synthesis of 3′-fluoro-2′,3′-dideoxy-2′,3′-didehydro-nucleosides (Scheme 1)

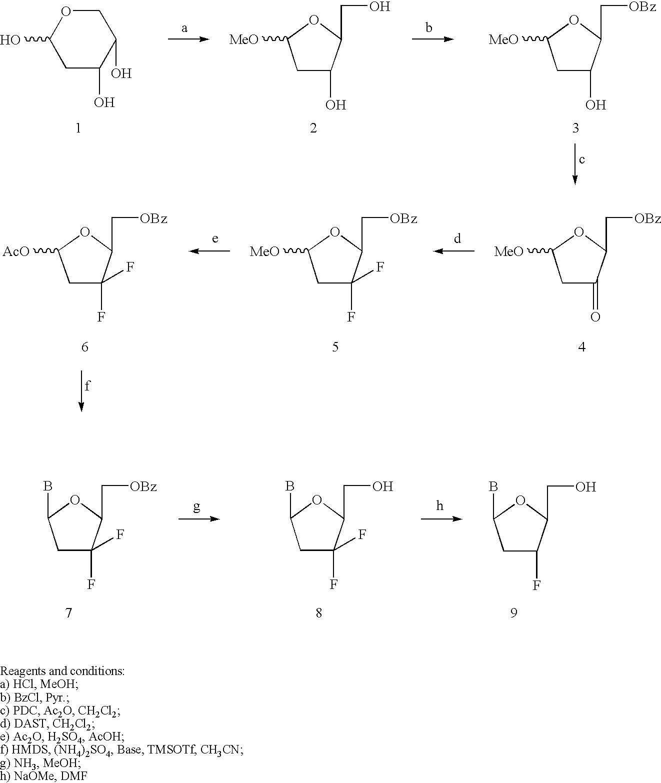

The 2′- and 3′-fluoro--2′,3--dideoxy-β-L-nucleosides, disclosed herein for the treatment of HIV and HBV infections in a host can be prepared according to published methods. β-L-nucleosides can be prepared from standard modifications of methods disclosed in, for example, the following publications: Chong, Y. et al., 2003, J. Med. Chem., 46, 3245-3256; Onuma, S. et al. 2002, Tethrahedron, 58:2497-2503; Jeong, et al., 1993, J. of Med. Chem., 36:182-195; European Patent Application Publication No. 0 285 884; Genu-Dellac et al., 1991, Antiviral Chem. Chemother. 2:83-92; Johansson, K. N. G. et al., European Patent Application 352 248; Mansuri, M. M. et al., 1991, Bioorg. Med. Chem. Lett. 1:65-68; Fujimori, S. et al., 1992, Nucleosides & Nucleotides 11:341-349; Holy A, 1992, Tetrahedron Lett., 2:189-192; Holy, A., 1992, Collect Czech Chem Commun. 37:4072-4087; Holy, A, 1992, Townsend L B, Tipson R S, ed. Nucleic Acid Chem. New York: Wiley, Vol. 1:347-353); Okabe, M., et al.; 1988, J Org Chem. 53:4780-4786; Robins, M. J., et al., 1992, J Org Chem. 35:363-639; Genu-Dellac, C. et al.; 1991, Tet Lett 32(1):79-82; Genu-Dellac, C. et al. 1991, 216:240-255; and Genu-Dellac, C., et al. 1991, 10(b): 1345-1376.

2. Synthesis of 3′-substituted-2′,3′-dideoxy-2′,3′-didehydro-nucleosides (Scheme 2)

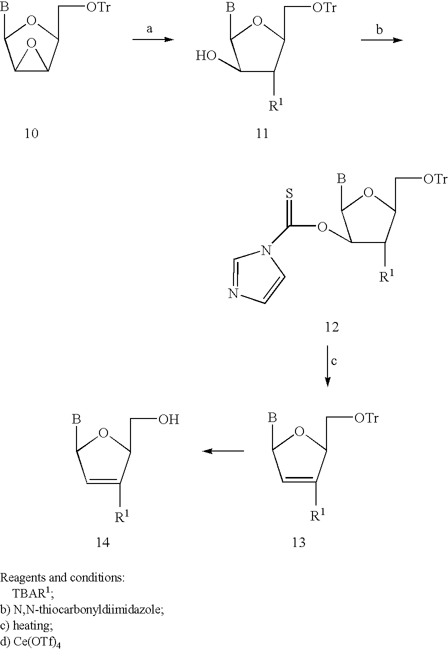

Other 3′-substituted-d4-nucleosides (14) can be prepared by similar methods as reported in Matsuda, A.; Satoh, M.; et al. Heterocycles, 1988, 27, 2545-2548; Faul, M. M.; et al Tetrahedron, 1999, 53, 8085-8104; and schematically shown above. R¹ can be N₃, CN, and alkyl groups.

Enzymatic methods for the separation of D and L enantiomers of cis-nucleosides are disclosed in, for example, but not limited to, Nucleosides and Nucleotides, 12(2), 225-236 (1993); European Patent Application Nos. 92304551.2 and 92304552.0 and PCT Publication Nos. WO 91/11186, WO 92/14729, and WO 92/14743. Separation of the acylated or alkylated racemic mixture of D and L enantiomers of cis-nucleosides can be accomplished by high performance liquid chromatography with selected chiral stationary phases, as disclosed, for example, in PCT Publication No. WO 92/14729.

Mono, di, and triphosphate derivatives of the active nucleosides can be prepared as described according to published methods. The monophosphate can be prepared according to the procedure of Imai et al. 1969, J. Org. Chem., 34(6):1547-1550. The diphosphate can be prepared according to the procedure of Davisson et al., 1987, J. Org. Chem. 52(9):1794-1801. The triphosphate can be prepared according to the procedure of Hoard et al., 1965, J. Am. Chem. Soc., 87(8): 1785-1788.

The antivirally active nucleosides can be administered as any derivative that upon administration to the host recipient, is capable of providing, directly or indirectly, the parent compound, or that exhibits activity itself. Nonlimiting examples include the pharmaceutically acceptable salts (alternatively referred to as “physiologically acceptable salts”) and prodrugs.

Modifications of the active compound, specifically at the N⁴ and 5′-O positions, can affect the bioavailability and rate of metabolism of the active species, thus providing control over the delivery of the active species. Further, the modifications can affect the antiviral activity of the compound, in some cases increasing the activity over the parent compound. This can easily be assessed by preparing the derivative and testing its antiviral activity according to the methods described herein, or other method known to those skilled in the art.

Stereoisomerism and Polymorphism

Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

The nucleosides of the present invention contain at least two critical chiral carbon atoms. In general, the substituents on the chiral carbons [the specified purine or pyrimidine base (referred to as the C1 substituent when using the sugar ring intermediate numbering) and CH₂OH (referred to as the C4 substituent)] of the nucleoside can be either cis (on the same side) or trans (on opposite sides) with respect to the sugar ring system. Both the cis and trans racemates consist of a pair of optical isomers. Hence, each compound has four individual stereoisomers. The four stereoisomers are represented by the following configurations (when orienting the sugar moiety in a horizontal plane such that the —O— moiety is in back): (1) cis, with both groups “up”, which is referred to as β-D; (2) the mirror image, i.e., cis, with both groups “down”, which is the mirror image is referred to as β-L; (3) trans with the C4 substituent “up” and the C1 substituent “down” (referred to as α-D); and (4) trans with the C4 substituent “down” and the C1 substituent “up” (referred to as α-L). The two cis enantiomers together are referred to as a racemic mixture of β-enantiomers, and the two trans enantiomers are referred to as a racemic mixture of α-enantiomers.

As known to those skilled in the art of nucleoside chemistry, in some cases, one of the β-cis enantiomers can be more active, or less toxic, than the other enantiomer. This can be easily determined by separating the enantiomers and testing the activity and cytotoxicity using standard assays.

Pharmaceutically Acceptable Salts and Prodrugs

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, but not limited to, tosylate, rnethanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by reference herein: Kucera, L. S., et al. 1990. AIDS Rex Hum. Retro Viruses. 6:491-501; Piantadosi, G., et al. 1991. J. Med. Chem. 34:1408.1414; Hosteller, K. Y., et al. 1992, Antim. Agents Chemother. 36:2025.2029; Hosetler, K. Y., et al. 1990, J. Biol. Chem. 265:61127.

Nonlimiting examples of U.S. patents that disclose suitable lipophilic substituents that can be covalently incorporated into the nucleoside, preferably at the 5′-OH position of the nucleoside or lipophilic preparations, include U.S. Pat. Nos. 5,149,794; 5,194,654; 5,223,263; 5,256,641; 5,411,947; 5,463,092; 5,543,389; 5,543,390; 5,543,391; and 5,554,728, all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

Pharmaceutical Compositions

Pharmaceutical compositions based upon a nucleoside compound of formula (I) and (II) or its pharmaceutically acceptable salt or prodrug can be prepared in a therapeutically effective amount for treating an HBV or HIV viral infection or abnormal cellular proliferation, optionally in combination with a pharmaceutically acceptable additive, carrier or excipient. The therapeutically effective amount may vary with the infection or condition to be treated, its severity, the treatment regimen to be employed, the pharmacokinetics of the agent used, as well as the patient treated.

In one aspect according to the present invention, the compound according to the present invention is formulated preferably in admixture with a pharmaceutically acceptable carrier. In general, it is preferable to administer the pharmaceutical composition in orally administrable form, but formulations may be administered via parenteral, intravenous, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification, etc.).

In certain pharmaceutical dosage forms, the prodrug form of the compound, especially including acylated (acetylated or other) and ether derivatives, phosphate esters and various salt forms of the present compounds, is preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound in the treatment of HBV and HIV viral infections.

The amount of compound included within therapeutically active formulations, according to the present invention, is an effective amount for treating the infection or condition, in preferred embodiments, an HBV or an HIV viral infection. In general, a therapeutically effective amount of the present compound in pharmaceutical dosage form usually ranges from about 0.1 mg/kg to about 100 mg/kg or more, depending upon the compound used, the condition or infection treated and the route of administration. For purposes of the present invention, a prophylactically or preventively effective amount of the compositions, according to the present invention, falls within the same concentration range as set forth above for therapeutically effective amount and is usually the same as a therapeutically effective amount.

Administration of the active compound may range from continuous (intravenous drip) to several oral administrations per day (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the seventy of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient.

For parenteral formulations, the carrier will usually comprise sterile water or aqueous sodium chloride solution, though other ingredients, including those that aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

In addition, compounds according to the present invention can be administered in combination or alternation with one or more antiviral, anti-HBV, anti-HIV or interferon, anti-bacterial agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

Combination or Alternation Therapy

In another embodiment, for the treatment, inhibition, prevention and/or prophylaxis of viral infection, the active compound or its derivative or salt can be administered in combination or alternation with another antiviral agent. In general, in combination therapy, effective dosages of two or more agents are administered together, whereas during alternation therapy, an effective dosage of each agent is administered serially. The dosage will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Nonlimiting examples of antiviral agents that can be used in combination with the compounds disclosed herein include, but are not limited to, acyclovir (ACV), ganciclovir (GCV or DHPG) and its prodrugs (e.g. valyl-ganciclovir), E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-α, pegelated interferon-α, lamivudine (3TC), LdT (or its prodrug), LdC (or its prodrug), tenofovir (or its prodrug), and adefovir (or its prodrug).

Without intent to limit the scope of the invention, exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, they should in no way, regardless of whether they are right or wrong, limit the scope of the invention so long as data are processed, sampled, converted, or the like according to the invention without regard for any particular theory or scheme of action.

### EXAMPLES

### Example 1

### Synthesis of the Active Compounds

Synthesis of 3′-substituted-2′,3′-didehydro-2′,3′dideoxy-β-L-nucleosides I, was accomplished by the method of Chong, Y. et al. 2003, J. Med. Chem., 46:3245-3256. Synthesis of 3′-Cl, I, Br, and alkyl substituted-2′,3′-dehydro-2′,3′-dideoxy-nuleoside can be achieved by the method of Onuma, S. et al. 2002, Tethrahedron, 58:2497-2503. Other 3′-substituted-d4-nucleosides can be prepared by a similar method as reported in Matsuda, A.; Satoh, M.; et al. Heterocycles, 1988, 27, 2545-2548; Faul, M. M.; et al Tetrahedron, 1999, 53, 8085-8104.

### Example 2

### Biological Activity of the Active Nucleosides

The HepAD38 (wild-type virus: rtM204) and HepAD79 (3TC-resistant virus: rtV204) (Stuyver L. J. et al. 2001, Hepatol., 33:751-757) cell lines replicate HBV under conditions that can be regulated with tetracycline (King R. W. et al. 2000, Methods in Molecular Medicine, Vol 24: Antiviral Methods and Protocols, 43-50 (eds: D. Kinchington & R. F. Schinazi), Humana Press Inc, Totowa, N.J.); Ladner, et al. 1997, Antimicrob. Agents Chemother., 41:1715-1720; Ladner S. K., et al. 1998a. Antivir. Chem. Chemother., 9:65-72; Ladner S. K., et al. 1998b. Antimicrob. Agents Chemother., 42:2128-31). In the presence of this drug, the cell supernatant is virtually free of viral DNA, but upon the removal of tetracycline from the culture medium, these cells secrete virus-like particle into the supernatant.

HepAD38 and HepAD79 cells were seeded at 5×10⁴ cells/well in a 96-well plate in seeding medium (DMEM/F12+10% FBS, 50 μg/ml penicillin, 50 μg/ml streptomycin, 100 μg/ml kanamycin, 400 μg/ml G418, and 0.3 μg/ml tetracycline) and incubated for 2 days at 37° C. in a 5% CO₂, humidified atmosphere. The seeding media was removed, and cells were washed 3 times with PBS. Cells were then incubated with 200 μL assay medium (DMEM/F12+10% FBS+P/S/K) containing either (i) no compound, (ii) test compound, or (iii) control drugs at 10 μM. After an additional 5-day (HepAD38) or 13-day incubation (HepAD79, with renewal of the culture media at day 7), the cell supernatant was collected and stored at −70° C. until HBV DNA was quantified.

Amplification of HBV DNA

HBV DNA was extracted from supernatant using QiaAmp DNA blood Mini kit (Qiagen) and nucleic acids eluted in 200 μl Viral DNA was detected by quantitative PCR (Q-PCR). The TaqMan probe and primers were designed using the Primer Express software (Applied Biosystems) and cover highly conserved sequences complementary to the DNA sequences present in HBsAg. A total of 5 μl DNA was amplified using reagents and conditions as described by the manufacturer (Applied Biosystems). The standard curve showed a dynamic range of at least 6 logs of viral load (not shown).

Time Course of Viral Induction

The time course of viral induction of HBV in HepAD38 cells was investigated. Following a 2-day HBV suppression (using tetracycline containing medium), and a 5-day period without viral suppression (tetracycline-free medium), the following observations were made: (i) immediately after seeding, viral DNA was detected in cell supernatant, but the quantity did not change over the three sampling points (day 0, day 1 and day 2 in seeding media), indicating that the HBV DNA sequences in the supernatant were derived from cell debris; (ii) tetracycline completely shut down HBV DNA expression; and, (iii) 5 days post induction, there was an increase of approximately 2.5 logs of HBV DNA in the cell supernatant.

Subsequently, the time course of viral induction in HepAD79 cells over a 13-day incubation period was investigated. Viral DNA accumulated continuously over the whole incubation period, with a maximal virus production of ˜4.5 loglo (data not shown). Two antiviral compounds (3TC, adefovir) were included as controls, but both only reduced the amount of virus production marginally (EC₉₀>10 μM for both compounds).

As the HepAD79 cells were reaching confluency at day 3 post-seeding, it was anticipated that a certain number of cells would be dead at the end of the 15-day experiment, or became detached from the tissue culture plate. In such cases, the viral DNA that was detected in the supernatant could be a derived from cellular contamination during the collection procedure, or from cell debris. Therefore, the DNA extracted from the cell supernatant was also amplified by Q-PCR for the presence of cellular DNA (the rRNA gene). Cellular DNA could not be detected (data not shown), suggesting that the detectable viral DNA is continuously expressed over the whole incubation period. The dynamic range for antiviral testing in medium throughput assay conditions at day 14, however, was found to be ˜2.5 log₁₀ viral copies per ml, since tetracycline did not shut down completely the virus production (data not shown).

Compound Evaluation

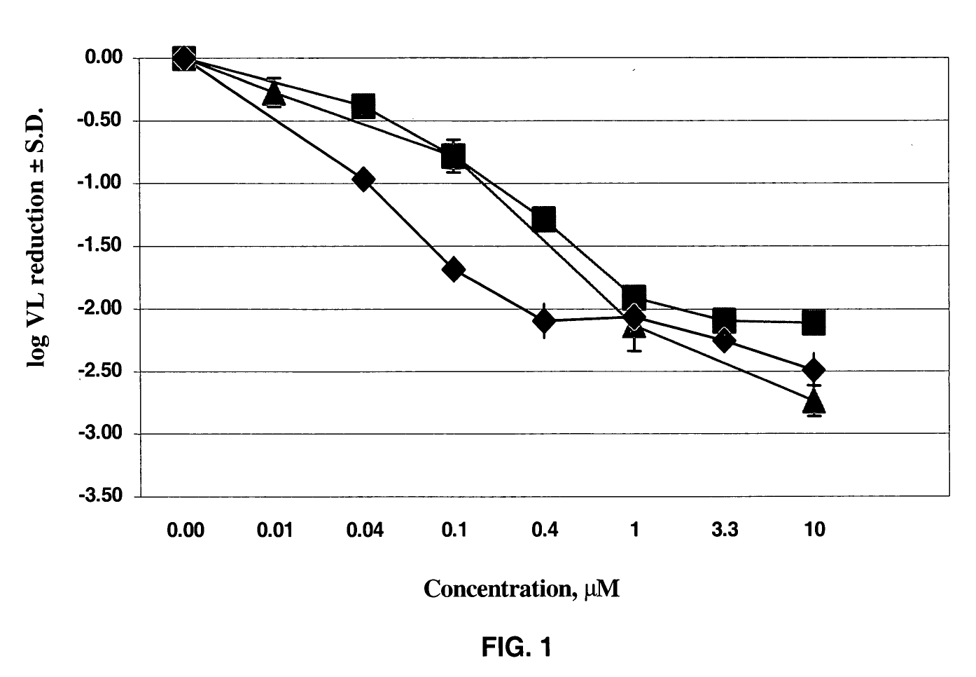

A series of molecules were unexpectedly discovered with potent anti-HBV activities in HepAD38 cells [L-3F-D4C (I (X=NH₂, Y=H, Z=0, R¹=F, R²=OH)) (effective concentration to reduce the viral load with 90% (EC₉₀)=0.25 μM), and L-3F-D4FC (I (X=NH₂, Y=F, Z=0, R¹=F, R²=OH)) (EC₉₀=0.25 μM). The data are depicted in FIG. 1 and Table 1 below. Potent inhibition of HBV production was also seen for 3TC (EC₉₀=0.05 μM). The β-L-3F-D4-compounds (I, R¹=F) are related to the previously discovered β-L-D4C (I (X=NH₂, Y=H, Z=0, R³′=H, R²=OH)) and β-L-D4FC (I (X=NH₂, Y=F, Z=0, R=H, R²=OH)) compounds (Lin T. S., et al. J. Med. Chem. 39:1757-9; Zhu Y. L., et al. Antimicrob. Agents Chemother. 42:1805-10; Ono S. K., et al. 2001, J. Clin. Invest. 107:449-55).

TABLE 1
Structure-Activity Relationship Of Compounds With Potent
Antiviral Activity Against HBV

| | HepAD38 | | HepAD79 | | FI EC₉₀ |
|---|---|---|---|---|---|
| | log₁₀ | | log₁₀ | | AD79/ |
| | inhibition | | inhibition | | EC₉₀ |
| | at 10 μM | EC₉₀ | at 10 μM | EC₉₀ | AD38 |
| 3TC | 1.97 ± 0.28 | 0.05 ± 0.02 | 0.67 ± 0.03 | >10 | >200 |
| Adefovir | 0.72 ± 0.12 | >10 | 0.91 ± 0.11 | ˜10 | |
| (−)-FTC | 1.59 ± 0.14 | 1.16 ± 0.66 | 0.28 | >10 | |
| (+)-FTC | 1.15 | 6.94 | 0.10 | >10 | |
| RCV | 1.61 ± 0.16 | 0.50 ± 0.16 | 0.05 | >10 | |
| L-FddC | 2.03 | 0.31 | 0.02 | >10 | |
| L-d4C | 2.79 ± 0.08 | 0.06 ± 0.02 | 1.54 ± 0.38 | 0.96 | 16 |
| L-d4FC | 2.56 ± 0.44 | 0.05 ± 0.02 | 1.64 ± 0.37 | 0.88 | 17.6 |
| L-2′F-D4C | 1.77 ± 0.13 | 0.74 | 0.33 | >10 | |
| L-2′F-D4FC | 1.91 ± 0.13 | 0.63 | 0.23 | >10 | |
| L-3′F-D4C | 2.68 ± 0.44 | 0.22 ± 0.15 | 1.65 ± 0.24 | 2.3 | 10.5 |
| L-3′F-D4FC | 2.8 ± 0.09 | 0.22 ± 0.14 | 1.56 ± 0.40 | 3.1 | 14.1 |

The same compounds were also evaluated for activity against 3TC-resistant virus, in terms of activity in HepAD79 cells (producing 3TC-resistant virus). Unexpectedly, the compounds β-L-D4C (EC₉₀=0.96 μM), β-L-D4FC (EC₉₀=0.88 μM), β-L-3′F-D4C (EC₉₀=2.3 μM) and β-L-3′F-D4FC (EC₉₀=3.1 μM) showed average fold reduction of 16.8 for the β-L-D4-compounds and average fold reduction of 12 for the β-L-3′F-D4-compounds (See Table 1).

The activity against single mutant HBV was previously reported for β-L-D4FC (Ono S. K., et al. J. Clin. Invest. 107:449-55). Contrary to the finding of the present invention, they reported that this compound had an EC₅₀ value of 1.8 μM for the M522V virus (which is identical to the rtV204, using the new nomenclature in Stuyver L. J., et al. 2001, Hepatol. 33:751-757).

In addition, L-2′F-D4C and L-2′F-D4FC were also found potent inhibitors of the wild type HBV, but became completely inactive against the mutant virus (Table 1). This observation is consistent with the previously held general belief that all β-L-compounds are cross-resistant with the rtV204 mutation.

### Example 3

### Toxicity Evaluation of the Active Nucleosides

The compounds summarized in Table 1 were tested for toxicity in several human cell lines. Compound β-L-3F-D4C (I (X=NH₂, Y=H, Z=0, R¹=F, R²=OH)) and β-L-3F-D4FC (I (X=NH₂, Y=F, Z=0, R=F, R=OH)) were found non-toxic in standard MTS assays (inhibitory concentration needed to reduce the cell metabolism by 50%, IC₅₀>100 μM). On the contrary, β-L-D4C (I (X=NH₂, Y=H, Z=0, R¹=H, R²=OH)) and β-L-D4FC (I (X=NH₂, Y=F, Z=0, R¹=H, R²=OH)) showed ED₅₀ concentrations (concentration required to inhibit 50% of cell growth) of 20 and 7 μM, respectively (Lin T. S. et al. 1996. J. Med. Chem. 39:1757-9).

TABLE 2
Relative Quantification Of Mitochondrial DNA In Hepg2 Cells After
14-Day Treatment With Modified β-L-D4-Compounds, Including
3TC And ddC As Controls

| | | | | Average | Fold difference |
|---|---|---|---|---|---|
| | | Average ΔCt ± | | Δ Δ Ct ± s.d. | in COXII DNA levels |
| | Conc | s.d. MitCoxII | | MitCOXII- | normalized for rDNA |
| Compound | (μM) | rDNA | | rDNA | (range) relative to control |
| No | | 0.00 ± 0.26 | 0.00 ± 0.57 | 0.00 ± 0.34 | 1.00 (1.27-0.80) |
| ddC | 10 | 7.70 ± 0.08 | 0.92 ± 0.52 | 6.78 ± 0.56 | 0.01 (0.01-0.01) |
| 3TC | 10 | 0.02 ± 0.10 | 0.48 ± 0.25 | 0.50 ± 0.23 | 0.70 (0.87-0.61) |
| β-L-D4C | 100 | 6.82 ± 0.33 | 10.14 ± 0.77 | −3.32 ± 0.75 | 9.99 (5.92-16.85) |
| | 10 | 2.72 ± 0.37 | 3.96 ± 0.87 | −1.24 ± 0.50 | 2.36 (1.67-3.35) |
| β-L-D4FC | 100 | 6.52 ± 0.14 | 8.76 ± 0.47 | 2.24 ± 0.58 | 4.72 (3.16-7.04) |
| | 10 | 2.72 ± 0.27 | 3.89 ± 0.23 | −1.17 ± 0.47 | 2.25 (1.62-3.12) |
| β-L-2′F-D4C | 100 | 0.33 ± 0.21 | 0.53 ± 0.60 | −0.20 ± 0.40 | 1.15 (0.87-1.52) |
| | 10 | −0.36 ± 0.02 | −0.06 ± 0.27 | −0.30 ± 0.28 | 1.23 (1.02-1.50) |
| β-L-2′F-D4FC | 100 | 0.83 ± 0.17 | 0.85 ± 0.51 | −0.02 ± 0.64 | 1.01 (0.65-1.58) |
| | 10 | 0.05 ± 0.07 | −0.23 ± 0.20 | 0.28 ± 0.17 | 0.82 (0.73-0.92) |
| β-L-3′F-D4C | 100 | 0.20 ± 0.20 | −0.28 ± 0.14 | 0.48 ± 0.13 | 0.72 (0.79-0.65) |
| | 10 | 0.39 ± 0.15 | −0.11 ± 0.38 | 0.49 ± 0.51 | 0.71 (1.02-0.50) |
| | 1 | 0.13 ± 0.16 | 0.08 ± 0.25 | 0.05 ± 0.26 | 0.96 (1.16-0.80) |
| β-L-3′F-D4FC | 100 | −0.52 ± 0.28 | −0.94 ± 0.16 | 0.42 ± 0.13 | 0.75 (0.82-0.68) |
| | 10 | 0.23 ± 0.13 | −0.10 ± 0.06 | 0.33 ± 0.10 | 0.80 (0.85-0.74) |
| | 1 | 0.00 ± 0.13 | −0.46 ± 0.06 | 0.35 ± 0.01 | 0.78 (0.79-0.78) |

In addition, these compounds were also tested in the HepG2 cell line (14-day treatment, 100 μM) for their ability to reduce mitochondrial DNA levels (using a newly designed normalized Q-PCR protocol as described in Stuyver, L. J., et al. 2002, Antimicrob. Agents Chemother., 46:3854-3860) or lactic acid production. The results of these experiments are shown in Table 2. The results of the present invention are in agreement with the observations that β-L-D4C (I(X=NH₂, Y=H, Z=0, R¹=H, R²=OH)) and β-L-D4FC (I (X=NH₂, Y=F, Z=O, R¹=H, R²=OH)) reduced the cell growth significantly, but they did not specifically affect the mitochondrial DNA levels (Lin T. S., et al. 1996, J. Med. Chem. 39:1757-9; Zhu Y. L., et al. 1998 Antimicrob. Agents Chemother., 42:1805-10). When tested at concentrations up to 100 μM for 14 days, β-L-3′ F-D4C (I (X=NH₂, Y=H, Z=0, R¹=F, R²=OH)) and β-L-3′ F-D4FC (I(X=NH₂, Y=F, Z=0, R=F, R=OH)) did not show any reduction in cell growth, and there were no reductions in mitochondrial DNA levels. There was no increase in total lactic acid production observed under those conditions (not tested for β-L-D4C (I (X=NH₂, Y=H, Z=O, R₃′=H, R²=OH)) and β-L-D4FC (I (X=NH₂, Y=F, Z=0, R³′=H, R²=OH)).

The above specification, examples and data provide a complete description of the manufacture and use of the composition of the invention. Since many embodiments of the invention can be made without departing from the spirit and scope of the invention, the invention resides in the claims hereinafter appended.

We claim:

1. A compound of the general formula: 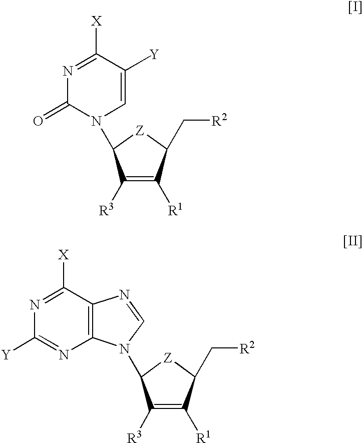
or its pharmaceutically acceptable salt or prodrug thereof,
wherein:
X is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R^{4.}
R² is OH, OR⁴, OC(O)R⁴, OPO₃H₂, OP₂O₆H₃, OP₃O₉H₄, OPO₃Na₂, OPO₃R⁴R⁵, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NHR⁴, NR⁴R⁵, NHOH, NHOR⁴, NHNH₂, NR⁴NH₂, NHNHR⁴, PO₃H₂, P₂O₆H₃, P₃O₉H₄, PO₃Na₂, P₂O₆Na₃, P₃O₉Na₄, PO₃R⁴R⁵, P₂O₆R⁴₂R⁵,
P₃O₉R⁴₃R⁵.
R³ is H.
Z is O, S, CH₂ or C═CH₂.
wherein R⁴ and R⁵ are the same or different and are lower alkane or alkene or acyl of carbon 1-17 or aryl or aralkyl, such as unsubstituted or substituted phenyl or benzyl.

2. The compound of claim 1 of the formula: 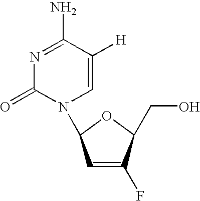
or its pharmaceutically acceptable salt or prodrug thereof.

3. The compound of claim 1 of the formula: 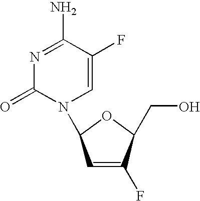
or its pharmaceutically acceptable salt or prodrug thereof.

4. A pharmaceutical composition for the treatment or prophylaxis of a hepatitis B viral infection in a host comprising an effective amount of a compound of the general formula: 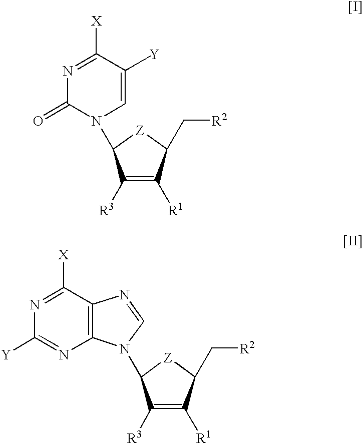
or its pharmaceutically acceptable salt or prodrug thereof,
wherein:
X is hydrogen, halogen (F, Cl, Br, I), NH₂, NHR⁴, NR⁴R⁵, NHOH, NHOR¹, NHNH₂, NR⁴NH₂, NHNHR⁴, SH, SR⁴, OH, OR⁴, N₃, CN, CF₃.
Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R^{4.}
R² is OH, OR⁴, OC(O)R⁴, OPO₃H₂, OP₂O₆H₃, OP₃O₉H₄, OPO₃Na₂, OPO₃R⁴R⁵, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NHR⁴, NR⁴R⁵, NHOH, NHOR⁴, NHNH₂, NR⁴NH₂, NHNHR⁴, PO₃H₂, P₂O₆H₃, P₃O₉H₄, PO₃Na₂, P₂O₆Na₃, P₃O₉Na₄, PO₃R⁴R⁵, P₂O₆R⁴₂R¹,
P₃O₉R⁴₃R⁵.
R³ is H.
Z is O, S, CH₂ or C═CH₂.
wherein R⁴ and R⁵ are the same or different and are lower alkane or alkene or acyl of carbon 1-17 or aryl or aralkyl, such as unsubstituted or substituted phenyl or benzyl;
optionally in a pharmaceutically acceptable carrier or diluent.

5. A pharmaceutical composition for the treatment or prophylaxis of a hepatitis B viral infection in a host comprising an effective amount of a compound of the formula: 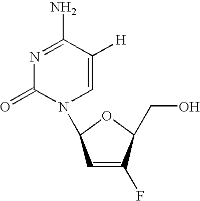
or its pharmaceutically acceptable salt or prodrug thereof;
optionally in a pharmaceutically acceptable carrier or diluent.

6. A pharmaceutical composition for the treatment or prophylaxis of a hepatitis B viral infection in a host comprising an effective amount of a compound of the formula: 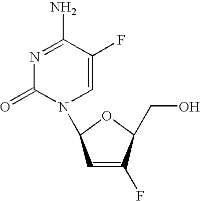
or its pharmaceutically acceptable salt or prodrug thereof; optionally in a pharmaceutically acceptable carrier or diluent.

7. The pharmaceutical composition of any of claims 4-6, wherein the hepatitis B virus is resistant to one or more other antivirally effective drugs.

8. The pharmaceutical composition of claim 7, wherein the hepatitis B virus is resistant to 3TC (lamivudine).

9. The pharmaceutical composition of claim 7, wherein the hepatitis B virus is resistant to (−)-FTC.

10. The pharmaceutical composition of any of claims 4-9, wherein the composition or its pharmaceutically acceptable salt or prodrug thereof is administered in combination or alternation with one or more other antivirally effective agents.

11. The pharmaceutical composition of claim 10, wherein the other antivirally effective agent is one or more agents selected from the group consisting of acyclovir (ACV), ganciclovir (GCV or DHPG), valyl-ganciclovir, E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-α, pegelated interferon-α, lamivudine (3TC), LdT, LdC, tenofovir and adefovir.

12. A pharmaceutical composition for the treatment or prophylaxis of an HIV viral infection in a host comprising an effective amount of a compound of the general formula: 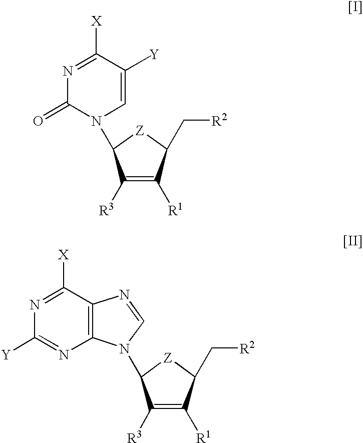
or its pharmaceutically acceptable salt or prodrug thereof,
wherein:
X is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R^{4.}
R² is OH, OR⁴, OC(O)R⁴, OPO₃H₂, OP₂O₆H₃, OP₃O₉H₄, OPO₃Na₂, OPO₃R⁴R¹, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NH₄, NR⁴R⁵, NHOH, NHOR⁴, NHNH₂, NR⁴NH₂, NHNHR^{4, PO}₃H₂, P₂O₆H₃, P₃O₉H₄, PO₃Na₂, P₂O₆Na₃, P₃O₉Na₄, PO₃R⁴R⁵, P₂O₆R⁴₂R⁵, P₃O₉R⁴₃R.
R³ is H.
Z is O, S, CH₂ or C═CH₂.
wherein R⁴ and R⁵ are the same or different and are lower alkane or alkene or acyl of carbon 1-17 or aryl or aralkyl, such as unsubstituted or substituted phenyl or benzyl;
optionally in a pharmaceutically acceptable carrier or diluent.

13. A pharmaceutical composition for the treatment or prophylaxis of an HIV viral infection in a host comprising an effective amount of a compound of the formula: 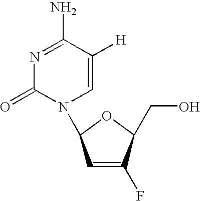
or its pharmaceutically acceptable salt or prodrug thereof;
optionally in a pharmaceutically acceptable carrier or diluent.

14. A pharmaceutical composition for the treatment or prophylaxis of an HIV viral infection in a host comprising an effective amount of a compound of the formula: 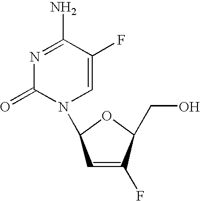
or its pharmaceutically acceptable salt or prodrug thereof;
optionally in a pharmaceutically acceptable carrier or diluent.

15. The pharmaceutical composition of any of claims 12-14, wherein the HIV virus is resistant to one or more other antivirally effective drugs.

16. The pharmaceutical composition of claim 15, wherein the HIV virus is resistant to 3TC (lamivudine).

17. The pharmaceutical composition of claim 15, wherein the IUV virus is resistant to (−)-FTC.

18. The pharmaceutical composition of any of claims 12-17, wherein the composition or its pharmaceutically acceptable salt or prodrug thereof is administered in combination or alternation with one or more other antivirally effective agents.

19. The pharmaceutical composition of claim 18, wherein the other antivirally effective agent is one or more agents selected from the group consisting of acyclovir (ACV), ganciclovir (GCV or DHPG), valyl-ganciclovir, E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)—HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-α, pegelated interferon-α, lamivudine (3TC), LdT, LdC, tenofovir and adefovir.

20. A method for treating a host infected with hepatitis B virus comprising administering an effective amount of a compound of the general formula: 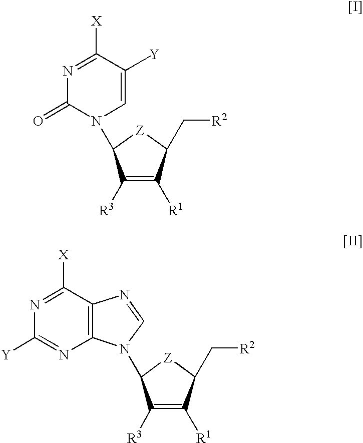
or its pharmaceutically acceptable salt or prodrug thereof,
wherein:
X is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R^{4.}
R² is OH, OR⁴, OC(O)R⁴, OPO₃H₂, OP₂O₆H₃, OP₃O₉H₄, OPO₃Na₂, OPO₃R⁴R¹, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NHR⁴, NR⁴R⁵, NHOH, NHOR⁴, NHNH₂, NR⁴NH₂, NHNHR⁴, PO₃H₂, P₂O₆H₃, P₃O₉H₄, PO₃Na₂, P₂O₆Na₃, P₃O₉Na₄, PO₃R⁴R⁵, P₂O₆R⁴₂R⁵, P₃O₉R⁴₃R^{5.}
R³ is H.
Z is O, S, CH₂ or C═CH₂.
wherein R⁴ and R⁵ are the same or different and are lower alkane or alkene or acyl of carbon 1-17 or aryl or aralkyl, such as unsubstituted or substituted phenyl or benzyl;
optionally in a pharmaceutically acceptable carrier or diluent.

21. The method of claim 20, wherein the compound or a pharmaceutically acceptable salt or prodrug thereof is administered in combination or alternation with one or more other antivirally effective agents.

22. The method of claim 21, wherein the other antivirally effective agent is one or more agents selected from the group consisting of acyclovir (ACV), ganciclovir (GCV or DHPG), valyl-ganciclovir, E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-α, pegelated interferon-α, lamivudine (3TC), LdT, LdC, tenofovir and adefovir.

23. The method of claim 22, wherein the compound of formula I or a pharmaceutically acceptable salt or prodrug thereof is selected from the group consisting of: 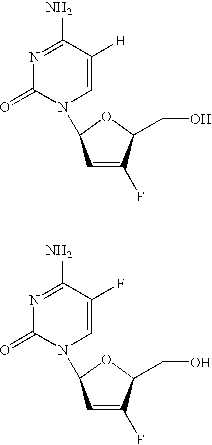
optionally in a pharmaceutically acceptable carrier or diluent.

24. The method of claim 23, wherein the compound or a pharmaceutically acceptable salt or prodrug thereof is administered in combination or alternation with one or more other antivirally effective agents.

25. The method of claim 24, wherein the other antivirally effective agent is one or more agents selected from the group consisting of acyclovir (ACV), ganciclovir (GCV or DHPG), valyl-ganciclovir, E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-α, pegelated interferon-α, lamivudine (3TC), LdT, LdC, tenofovir and adefovir.

26. The method of any of claims 20-25, wherein the hepatitis B virus is resistant to one or more other antivirally effective drugs.

27. The method of claim 26, wherein the hepatitis B virus is resistant to 3TC (lamivudine).

28. The method of claim 26, wherein the hepatitis B virus is resistant to (−)-FTC.

29. A method for treating a host infected with an HIV virus comprising administering an effective amount of a compound of the general formula: 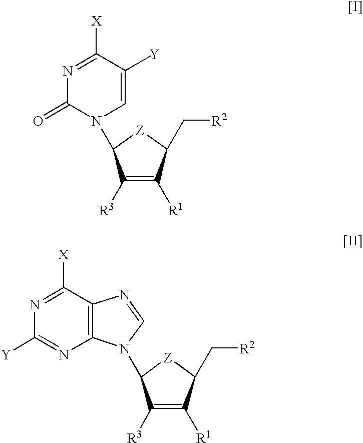
or its pharmaceutically acceptable salt or prodrug thereof,
wherein:
X is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
Y is hydrogen, halogen (F, Cl, Br, I), NH₂, NHR⁴, NR⁴R⁵, NHOH, NHOR⁴, NHNH₂, NR⁴NH₂, NHNHR⁴, SH, SR⁴, OH, OR⁴, N₃, CN, CF₃.
R¹ is H, halogen (F, Cl, Br, I), CN, CF₃, N₃, CH₃, CH₂CH₃, C(N₃)═CH₂, CH₂OH, CH═CH₂, ethynyl, CONH₂, CSNH₂, COOH, COOR⁴, or R^{4.}
R² is OH, OR⁴, OC(O)R⁴, OPO₃H₂, OP₂O₆H₃, OP₃O₉H₄, OPO₃Na₂, OPO₃R⁴R⁵, OP₂O₆Na₃, OP₂O₆R⁴₂R⁵, OP₃O₉Na₄, OP₃O₉R⁴₃R⁵, SH, SR⁴, SC(O)R⁴, NH₂, NHC(O)R⁴, NHR⁴, NR⁴R⁵, NHOH, NHOR⁴, NHNH₂, NR⁴NH₂, NHNHR⁴, PO₃H₂, P₂O₆H₃, P₃O₉H₄, PO₃Na₂, P₂O₆Na₃, P₃O₉Na₄, PO₃R⁴R⁵, P₂O₆R⁴₂R⁵, P₃O₉R⁴₃R⁵.
R³ is H.
Z is O, S, CH₂ or C═CH₂.
wherein R⁴ and R⁵ are the same or different and are lower alkane or alkene or acyl of carbon 1-17 or aryl or aralkyl, such as unsubstituted or substituted phenyl or benzyl;
optionally in a pharmaceutically acceptable carrier or diluent.

30. The method of claim 29, wherein the compound or a pharmaceutically acceptable salt or prodrug thereof is administered in combination or alternation with one or more other antivirally effective agents.

31. The method of claim 30, wherein the other antivirally effective agent is one or more agents selected from the group consisting of acyclovir (ACV), ganciclovir (GCV or DHPG), valyl-ganciclovir, E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-(x, pegelated interferon-(x, lamivudine (3TC), LdT, LdC, tenofovir and adefovir.

32. The method of claim 29, wherein the compound of formula I or a pharmaceutically acceptable salt or prodrug thereof is selected from the group consisting of: 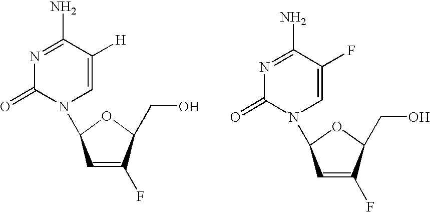
optionally in a pharmaceutically acceptable carrier or diluent.

33. The method of claim 32, wherein the compound or a pharmaceutically acceptable salt or prodrug thereof is administered in combination or alternation with one or more other antivirally effective agents.

34. The method of claim 33, wherein the other antivirally effective agent is one or more agents selected from the group consisting of acyclovir (ACV), ganciclovir (GCV or DHPG), valyl-ganciclovir, E-5-(2-bromovinyl)-2′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxypropyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)cytosine [(S)-HPMPC, or cidofivir], and (2S,4S)-1-[2-(hydroxymethyl)-1,3-dioxolan-4-yl]-5-iodouracil (L-5-IoddU), FTC, entecavir, interferon-α, pegelated interferon-α, lamivudine (3TC), LdT, LdC, tenofovir and adefovir.

35. The method of any of claims 29-34, wherein the HIV virus is resistant to one or more other antivirally effective drugs.

36. The method of claim 35, wherein the HIV virus is resistant to 3TC (lamivudine).

37. The method of claim 35, wherein the HIV virus is resistant to (−)-FTC.
